(19) United States
(12) Patent Application Publication
(10) Pub. No.: US 2024/0059925 A1
(43) Pub. Date: Feb. 22, 2024

(54) SUGAR-BASED, ENVIRONMENTALLY-FRIENDLY PIGMENT AND MINERAL DISPERSANTS

(71) Applicant: Colonial Chemical, Inc., South Pittsburg, TN (US)

(72) Inventors: Garret Bryant, Rocky Face, GA (US); Robert N. Comber, South Pittsburg, TN (US)

(21) Appl. No.: 18/268,585

(22) PCT Filed: Dec. 17, 2021
(86) PCT No.: PCT/US21/64158

§ 371 Date: Jun. 20, 2023

Related U.S. Application Data
(60) Provisional application No. 63/127,064, filed on Dec. 17, 2020.

(51) Int. Cl. C09D 105/00; C09D 7/41; C09D 7/65; C09D 7/20; C09D 7/61

(52) U.S. Cl. CPC C09D 105/00; C09D 7/41; C09D 7/65; C09D 7/20; C09D 7/61

(57) ABSTRACT

Pigment dispersion compositions within a liquid medium, ethylene oxide and 1,4-dioxane free, with low viscosity and moderate to high pigment concentrations using derivatized alkyl polyglucosides and/or alkyl polyglucoside sorbitan ester crosspolymers as dispersing agents

### PRIOR APPLICATIONS

This application claims benefit to U.S. patent application Ser. No. 63/127,064, filed on Dec. 17, 2020, the contents of which are incorporated herein by reference.

### BACKGROUND OF THE INVENTION

Pigments have been manufactured and used for hundreds of years. Before being used they are typically dispersed in a liquid medium for ease of application. Traditionally a type of oil or water has been used as the liquid medium, and the problem of dispersing certain pigments within liquids has always been a challenge. Natural pigments based on minerals can usually be dispersed with little difficulty, however, because of price synthetic alternatives have been created. These pigment alternatives can be impossible to properly disperse in a liquid medium without the addition of an agent, typically a surfactant, to facilitate the wetting and dispersing of the pigment. Likewise, a wetting or dispersing agent can be added when using natural pigments to maintain low viscosity of the blend. This allows the manufacturer to create more highly concentrated pigment dispersions.

Historically, many types of dispersing agents have been used to accomplish this task. Examples include carboxylic acids, phosphate esters, phosphate salts such as TKPP, and more recently polymeric surfactants based on acrylic or methacrylic acid; with the latter being the most common.

It is herein proposed that a new class of natural surfactants derived from alkylpoly glucosides can be used for the dispersion of pigments within a liquid medium to form dispersions of low viscosity and high concentrations.

### SUMMARY OF VARIOUS EMBODIMENTS OF THE INVENTION

One embodiment of the present invention is a composition for an aqueous pigment dispersion, comprising derivatized alkyl polyglucosides.

Another embodiment of the present invention is a crosspolymer of an alkyl polyglucoside.

Another embodiment of the present invention is the use of derivatized alkyl polyglucosides as dispersants for pigments, filler minerals, or functional minerals.

Another embodiment of the present invention is a dispersant for the following pigments/filler minerals/functional minerals including, but not limited to, phthalocyanine pigments, calcium carbonate, titanium dioxide, inorganic clays, iron oxides, ultramarine pigments, han purple, cobalt violet, manganese violet, Persian blue, cobalt blue, cerulean blue, Egyptian blue, manganese blue, azurite, copper carbonate, chrome green, terra verte, primrose yellow, crocoite, aurelian, yellow ochre, zinc chromate, raw umber, raw sienna, carbon black, zinc oxide, alizarin, arylide yellow, azo pigments, diarylide yellow, naphthol red, Indian yellow, indigo dye, orange 15, violet 23, yellow 10, yellow 12, yellow 13, yellow 16, yellow 81, yellow 83, yellow 139, yellow 185, quinacridone, tyrian purple, paris green, other organic or inorganic pigments, and other filler minerals.

Another embodiment of the present invention is a method of pigmenting, or adding filler, or structural minerals to latex paint formulations

Another embodiment of the present invention is a paint composition that comprises a dispersant of the present invention.

Another embodiment of the present invention is an adhesive composition that comprises a dispersant of the present invention.

Another embodiment of the present invention is a textile compound composition that comprises a dispersant of the present invention.

Another embodiment of the present invention is a water-resistant sealer composition that comprises a dispersant of the present invention.

Another embodiment of the present invention is an adhesion promoting primer composition that comprises a dispersant of the present invention.

Another embodiment of the present invention is a clear protective, or aesthetic clearcoat composition that comprises a dispersant of the present invention.

Another embodiment of the present invention is composition to be used as an additive for cementitious products, such as, but not limited to, cement, self-levelers, grouts, and mortars that comprises a dispersant of the present invention.

Another embodiment of the present invention is an additive for paper processing, specifically, pigmenting.

Another embodiment of the present invention is a personal care composition that comprises a dispersant of the present invention.

Another embodiment of the present invention is a solid polymer product composition that comprises a dispersant of the present invention.

Another embodiment of the present invention is a composition for an aqueous pigment dispersion that comprises at least one dispersant, such as derivatized alkyl polyglucoside, in a range of 0.01-50%; water, in a range from 20-80% by weight; at least one pigment for making up 20-80% of the dispersion.

In another embodiment of the present invention, the composition is ethylene oxide and 1,4-dioxane free.

In another embodiment of the present invention, the derivatized alkyl polyglucoside is chosen from a carboxymethyl derivatized alkyl polyglucoside, a quaternary derivatized alkyl poylglucoside, a sulfonate derivatized alkyl polyglucoside, a phosphate derivatized alkyl polyglucoside, a sulfosuccinate derivatized alkyl polyglucoside, a glycinate derivatized alkyl polyglucoside, and a citrate derivatized alkyl polyglucoside.

In another embodiment of the present invention, the derivatized alkyl polyglucoside is chosen from a polysulfonate derivatized alkyl polyglucoside, polyphosphate derivatized alkyl polyglucoside, polyquaternary derivatized alkyl polyglucoside, polycarboxylated derivatized alkyl polyglucoside, and a polycitrate derivatized alkyl polyglucoside

In another embodiment of the present invention, the dispersant is a sorbitan oleate decylglucoside crosspolymer. It should be understood that other crosspolymers can include other alkyl polyglucosides (for example, lauryl polyglucoside), and other sorbitan esters (for example, sorbitan laurate, sorbitan stearate, sorbitan myristate, and sorbitan palmitate).

In another embodiment of the present invention, the derivatized polyglucoside dispersant of the present invention comprises a monosaccharide unit, a disaccharide unit, a linker, and a functionalizing agent.

In another embodiment of the present invention, the derivatized polyglucoside dispersant of the present invention comprises a monosaccharide unit, a disaccharide unit, a linker, a crosslinking agent, and a functionalizing agent.

In another embodiment of the present invention, the composition further comprises a co-dispersant.

Another aspect of the present invention includes a process for producing a pigment dispersion. For example, the process may comprise providing an aqueous or organic solution that comprises at least one derivatized alkyl polyglucoside surfactant or one crosspolymer of an alkyl polyglucoside, water, and at least one pigment, filler mineral, or functional mineral.

Another embodiment of the present invention is a coating composition that comprises at least one derivatized alkyl polyglucoside dispersant and/or alkyl polyglucoside sorbitan ester crosspolymer of the present invention; water, and/or other organic solvent; a polymer and/or alkyd; a filler and/or functional mineral; and at least one colorant or pigment.

In another embodiment of the present invention, the pigment dispersant is used in a paint, adhesive, primer composition, textile composition, clearcoat composition, water resistant sealer, cementitious product, paper binder, alkyd emulsion, or personal care product; and is incorporated alone or through a pigment dispersion in which it is contained.

In another embodiment of the present invention, the personal care product is a cream, lotion, hair styling product, hair or skin conditioner, body wash, facial wash, shampoo, hair dye, color cosmetic, or any product making use of pigments, filler minerals, or functional minerals.

### BRIEF DESCRIPTION OF THE FIGURES

FIG. 1 is a graph that shows dispersant demand viscosity curves for yellow iron oxide in various derivatized alkyl polyglucosides and APG crosspolymers of the present invention.

FIG. 2 is a graph that shows dispersant demand viscosity curves for phthalo blue in various APG crosspolymers of the present invention.

FIG. 3 is a graph that shows dispersant demand viscosity curves for titanium dioxide in various APG crosspolymers of the present invention.

When described herein, Poly Suga®Mulse D6 and Poly Suga® Mulse D9 refer to a sorbitan oleate decylglucoside crosspolymer; Suga®Nate 160NC is a sodium laurylglucosides hydroxypropylsulfonate; PolySuga®Nate 160P is a sodium hydroxypropyl sulfonate laurylglucoside crosspolymer; and, PolySuga®Phos 8600P is a sodium hydroxypropyl phosphate cocoglucoside crosspolymer. All are manufactured and sold by Colonial Chemical, Inc., South Pittsburg, TN.

### DETAILED DESCRIPTION OF THE INVENTION

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which need to be independently confirmed.

The compositions of the present invention are many times mixtures of alkyl polyglucosides, and optionally a linker arm, and optionally a functionalizing agent, and optionally a crosslinking agent, which are often characterized as constitutional isomers. Constitutional isomers are compounds that have the same general empirical formula but differ in their constitution, i.e. in their structure, such that they can have a different sequence of the atoms and/or different bonds. Constitutional isomers are therefore fundamentally different from stereoisomers, which include both enantiomers and diastereomers.

Constitutional isomers are in many cases grouped into functional isomers, skeletal isomers, positional isomers and bonding isomers. In the case of functional isomers and bonding isomers, the compounds can have different reactivity; for example, ethanol comprises a hydroxyl group, whereas the constitutionally isomeric dimethyl ether has an ether group. Skeletal isomers and positional isomers differ in the branching and/or the position of functional groups, such that these constitutional isomers can have essentially the same functionality. The expression “essentially the same functionality” accordingly means that the underlying functional group, i.e., for example, a hydroxyl group, a phenyl ring or an ester group, is present in all constitutional isomers, but does not take account of altered reactivity of these groups as a result of different substitution. For example, there is a measurable difference in the reactivity of 1-n-butanol and tert-butanol owing to the stereochemistry, but the functionality as such is identical. In this connection, however, these measurable differences that are covered by the term “essentially the same functionality” are to be neglected, since both compounds in the present case have a hydroxyl functionality. On the other hand, propyne has one alkyne functionality and propadiene has two alkene functionalities. Alkenes, by comparison with alkynes, have a different functionality in the context of this invention, since they exhibit different acidity, for example. Therefore, propyne, by comparison with propadiene, does not have “essentially the same functionality”.

The mixtures of the present derivatized alkyl polyglucosides have essentially the same functionality. Accordingly, components of the mixture, while constitutional isomers, are not functional isomers, and instead are skeletal isomers and/or positional isomers. That is, the functional group may be in a different position on the same carbon chain or on the same sugar molecule and have essentially the same functionality.

The term “alkyl” refers to a straight or branched chain monovalent hydrocarbon radical having a specified number of carbon atoms. Alkyl groups may be unsubstituted or substituted with substituents that do not interfere with the specified function of the composition and may be substituted once or twice with the same or different group. Substituents may include alkyl, aryl, alkoxy, hydroxy, mercapto, amino, alkyl substituted amino, nitro, carboxy, carbanyl, carbanyloxy, cyano, methylsulfonylamino, or halogen, for example. Examples of “alkyl” include, but are not limited to, methyl, ethyl, n-propyl, isopropyl, n-butyl, s-butyl, t-butyl, n-pentyl, n-hexyl, 3-methylpentyl, and the like.

The term “surfactant”, “surface active agent”, “dispersant”, or “dispersing agent” refers to an organic chemical that when added to a liquid changes the properties of that liquid at a surface. In this instance they modify the wetting properties of pigments, and/or minerals as well as influences the pigment particles interaction with other particles, influencing their aggregation and stability in a liquid carrier.

Embodiments of the present invention are ethylene oxide and 1,4-dioxane free.

Alkyl polyglucosides are complex products made by the reaction of glucose and fatty alcohol. In dealing with the chemistry one talks about degree of polymerization (the so called “d.p.”). In the case of traditional alkyl poluglycosides the d.p. is around 1.4. This means that on average the is 1.4 units of glucose for each alkyl group. The fact of the matter is that the resulting material is a mixture having an average of 1.4.

The specific structure of the product is hard to ascertain completely since many positional isomers are possible, but two examples of structures are as follows:

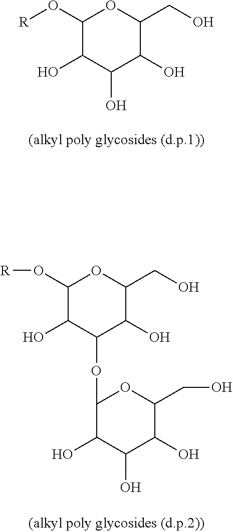

It should be clear that if there is a 50/50 mixture of the d.p. 1 and d.p. 2 product, the resulting analytical data will show that on average there is a d.p. of 1.5. Saying that a molecule has a d.p. of 1.5 does not mean that each molecule has 1.5 glucose units on it.

In one embodiment of the present invention, the dispersant is one that is disclosed in U.S. Pat. No. 6,627,612, incorporated herein by reference; and/or dispersants sold by Colonial Chemical, Inc. under the brand names Suga®Nate and Suga®Fax.

Another embodiment of the present invention is a dispersant disclosed in U.S. Pat. No. 6,958,315, incorporated herein by reference; and/or dispersants sold by Colonial Chemical, Inc. under the brand name Suga®Glycinate.

Another embodiment of the present invention is a dispersant disclosed in U.S. Pat. No. 8,268,766, incorporated herein by reference; and/or dispersants sold by Colonial Chemical, Inc. under the brand name Poly Suga®Mulse.

Another embodiment of the present invention is a dispersant disclosed in U.S. Pat. No. 7,507,399, incorporated herein by reference; and/or dispersants sold by Colonial Chemical, Inc. under the brand names Poly Suga®Quats, PolySuga®Nates, PolySuga®Phos.

Another embodiment of the present invention is a dispersant disclosed in U.S. Pat. No. 7,087,571, incorporated herein by reference; and/or dispersants sold by Colonial Chemical, Inc. under the brand name Suga®Mates.

Another embodiment of the present invention is a dispersant disclosed in U.S. Pat. No. 7,335,627, incorporated herein by reference; and/or dispersants sold by Colonial Chemical, Inc. under the brand name Poly Suga®Carb.

Other embodiments of the present invention include dispersants that are sugar-based sulfonate-, phosphate-, glycinate-, sulfosuccinate-, and carboxylate-containing dispersants derived from alkyl polyglucosides, including those disclosed in U.S. Patent Nos. 6,627,612; 6,958,315; 7,087,571; 7,507,399 and 7,335,627.

As stated above, the embodiment of the present invention are particularly effective dispersants for the dispersion, wetting, and stability of pigments and/or minerals in a liquid carrier. These embodiments are also particularly effective dispersants for the addition of pigments as a liquid slurry to products made with latex such as paints, or other coatings. They are also effective dispersants to be used in personal care, or cosmetics formulations. They also work alone or in combination with nonionic, alkyl polyglucoside-based sorbitan-ester crosspolymers (including those disclosed in U.S. Pat. No. 8,268,766). The latter crosspolymers of sorbitan esters and APG's are also very effective dispersants, wetting and stability of pigments and/or minerals in a liquid carrier.

The compositions of the present invention include a single embodiment, or mixture, used alone or in combination with an additional embodiment. The additional embodiment can be in the role of a co-dispersant

Other co-dispersants can be included in the mixtures of the present invention. Examples of the co-dispersants include ionic and nonionic dispersants.

These derivatized alkyl polyglucosides are naturally derived, do not possess polyoxyethylene groups (or contain residual ethylene oxide monomer or 1,4-dioxane), are biodegradable and in many cases have been found to have very low skin and eye irritation.

In one embodiment the derivatized alkyl polyglucoside is chosen from a carboxymethyl derivatized alkyl polyglucoside, a quaternary derivatized alkyl poylglucoside, a sulfonate derivatized alkyl polyglucoside, a phosphate derivatized alkyl polyglucoside, a sulfosuccinate derivatized alkyl polyglucoside, a glycinate derivatized alkyl polyglucoside, and a citrate derivatized alkyl polyglucoside.

In another embodiment of the present invention, the derivatized alkyl polyglucoside is chosen from a polysulfonate derivatized alkyl polyglucoside, polyphosphate derivatized alkyl polyglucoside, polyquaternary derivatized alkyl polyglucoside, polycarboxylated derivatized alkyl polyglucoside, and a polycitrate derivatized alkyl polyglucoside.

In another embodiment of the present invention, the dispersant is a sorbitan ester alkylglucoside crosspolymer, more specifically a sorbitan ester oleate decylglucoside crosspolymer.

In another embodiment of the present invention, the dispersant is an alkoxylated alkyl polyglucoside as described in U.S. Pat. No. 6,800,741, incorporated herein by reference.

In one embodiment of the present invention, the derivatized polyglucoside dispersant of the present invention comprises a monosaccharide unit, a disaccharide unit, a linker, and a functionalizing agent.

In this regard, a derivatized alkyl polyglucoside composition of the present invention includes the following, as a mixture:

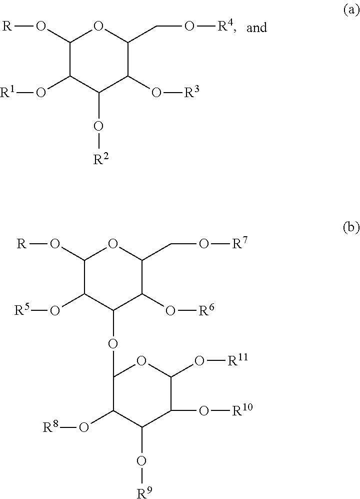

wherein:
R is an alkyl chain having 8 to 22 carbon atoms
R¹, R², R³, R⁴, R⁵, R⁶, R⁷, R⁸, R⁹, R¹⁰, and R¹¹ are independently selected from the group consisting of: —CH₂—C(O)—O⁻M⁺, or —C(O)CH₂—C(O)—O⁻M⁺

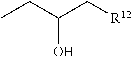

and H, with the proviso that R¹-R¹¹ are not all H;
R¹² is selected from the group consisting of:
—OH, —SO₃⁻M⁺, and —SO₄⁻²M⁺, —O—P(O)—(OM)₂,
—N(CH₃)₂—R^{1A}, —O—C(O)—CH₂—OH(SO₃⁻M⁺)—C(O)—O⁻M⁺,

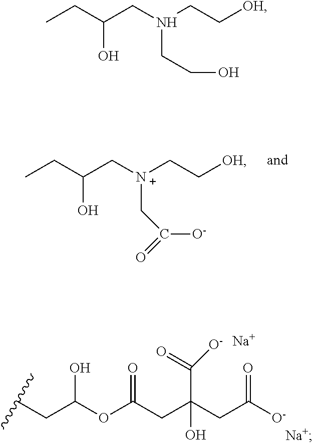

R^{1A} is CH₃—(CH₂)ₙ—;
M is a charge balancing group selected from H, Na, K, or NH₄⁺; and
n is an integer from 0-36;
and positional isomers thereof.

In one embodiment of the present invention, the derivatized polyglucoside dispersant of the present invention comprises a monosaccharide unit, a disaccharide unit, a crosslinking agent, and a functionalizing agent.

In this regard, a derivatized alkyl polyglucoside composition of the present invention includes the following, as a mixture:

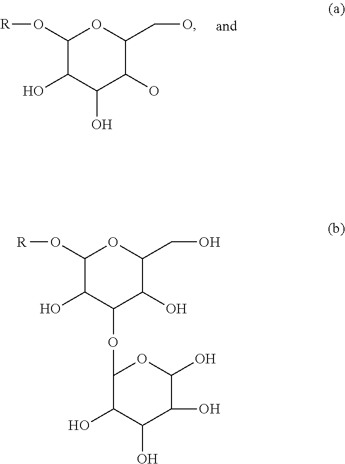

wherein:
R is an alkyl chain having 8 to 22 carbon atoms;
a crosslinking agent; and
a functionalizing agent selected from:

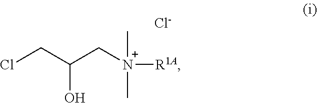

Cl—CH₂—CH(OH)—SO₃M, Cl—CH₂—CH(OH)—SO₄M, Cl—CH₂—CH(OH)—CH₂—OP(O)—(OM)₂, and combinations thereof, wherein
R^{1A} is CH₃(CH₂)ₙ—;
(ii) —Cl—CH₂—C(O) ⁻Nat⁺, 2-halocarboxylic acid, α, β-unsaturated carboxylic acid, cyclic carboxylic acid anhydride, and combinations thereof;

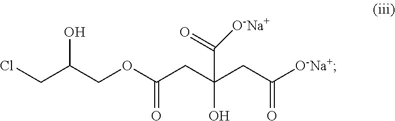

M is a charge balancing group selected from H, Na, K, or NH₄⁺; and
n is an integer from 0-36;
   and positional isomers thereof.

Crosslinking agents for the polyfunctionalized alkyl polyglucosides include those described immediately above include, but are not limited to, 1,3-dichloro-2-propanol and epichlorohydrin.

Thus, in one embodiment of the present invention is a phosphate and/or sulfonate functionalized alkyl polyglucoside of the following compounds, as a mixture, are useful as pigments dispersants, wetting agents, and stabilizers:

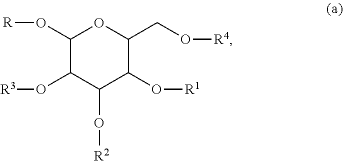

wherein:
R is an alkyl chain having 8 to 22 carbon atoms;
R¹, R², R³, and R⁴ are independently selected from the group consisting of:

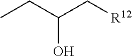

and H, with the proviso that R¹, R², R³, and R⁴ are not all H;
R¹² is selected from the group consisting of:
—OH, —SO₃⁻M⁺, —SO₄⁻²M⁺, and —O—P(O)—(OM)₂;
M is selected from the group consisting of Na, K, NH⁴; and

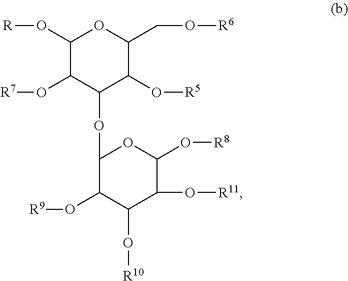

wherein
R⁵, R⁶, R⁷, R⁸, R⁹, R¹⁰, and R¹¹ are independently selected from the group consisting of:

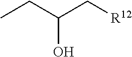

### and H; and

R¹² is selected from the group consisting of:
—OH, —O—P(O)—(OM)₂, —SO₃⁻M⁺, and —SO₄⁻²M⁺, and M is selected from the group consisting of Na, K, NH⁴;
   and positional isomers thereof.

These alkyl polyglucoside dispersants are manufactured by Colonial Chemical, Inc., South Pittsburg, TN 37380. Two examples of which are sodium laurylglucosides hydroxypropylsulfonate (sold under the brand name Suga®Nate 160NC) and sodium decylglucosides hydroxypropylsulfonate (sold under the brand name Suga®Nate 100NC). The alkylpolyglucoside phosphates of the current invention are manufactured by Colonial Chemical, In., South Pittsburg, TN 37380. An example of which is Sodium Decylglucosides Hydroxypropyl Phosphate, sold under the brand name Suga®Fax D10NC.

These dispersants are synthesized by the methods outlined in U.S. Pat. No. 6,627,612 or their corresponding patents and are generally supplied as clear solutions, 30-50% solids, that are used as pigment or mineral dispersants.

The phosphate functionalized alkyl polyglucoside dispersants of this embodiment are also described in U.S. Pat. No. 8,216,994. Thus, phosphate functionalized alkyl polyglucosides of the present invention include those with the following formula:

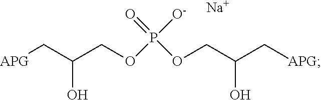

wherein APG is alkyl polyglucoside; and positional isomers thereof In some embodiments, the alkyl moiety contains about 12 carbon atoms. An example of a suitable phosphate functionalized alkyl polyglucoside includes, but is not limited to, sodium dilaurylglucoside hydroxypropyl phosphate.

The sulfonated functionalized alkyl polyglucoside dispersants of this embodiment are also described in U.S. Pat. No. 8,216,988. Thus, sulfonated functionalized alkyl polyglucosides of the present invention include those with the following formula:

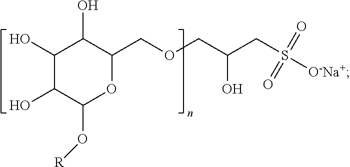

wherein n is between 1 to about 3, and particularly 1.5; and positional isomers thereof. R is an alkyl chain. Examples of suitable sulfonated functionalized alkyl polyglucosides include sodium laurylglucosides hydroxypropyl sulfonate and sodium declyglucosides hydroxypropyl sulfonate and combinations thereof.

An additional embodiment of the present invention is also a glycinate-modified alkylpolyglucoside dispersants represented by compounds of the following formulae, and positional isomers thereof, as a mixture:

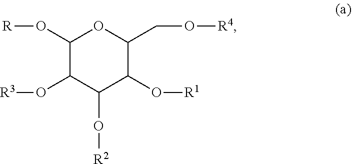

wherein
R is alkyl having 1 to 22 carbon atoms;
R¹, R², R³, and R⁴ are independently selected from

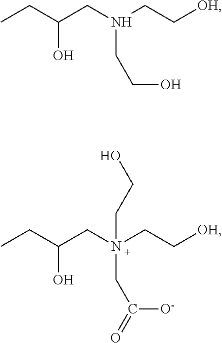

H, with the proviso that R¹, R², R³, and R⁴ are not all H; and

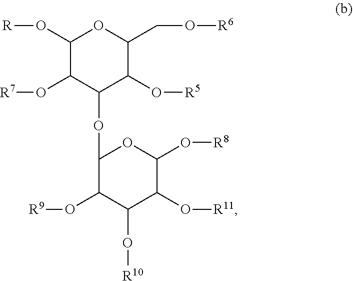

wherein
R is alkyl having 8 to 22 carbon atoms;
R⁵, R⁶, R⁷, R⁸, R⁹, R¹⁰, and R¹¹ are independently selected from

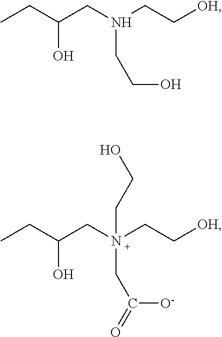

and H, with the proviso that R⁵, R⁶, R⁷, R⁸, R⁹, R¹⁰, and R¹¹ are not all H;
and positional isomers thereof.

The alkylpolyglucoside glycinates of the current invention are manufactured by Colonial Chemical, Inc., South Pittsburg, TN 37380, two examples of which are Sodium Bis-Hydroxyethylglycinate Coco-Glucosides Crosspolymer (sold under the brand name Poly Suga®Glycinate C) and Sodium Bis-Hydroxyethylglycinate Lauryl-Glucosides Crosspolymer (sold under the brand name Poly Suga®Glycinate L).

These dispersants are synthesized by the methods outlined in U.S. Pat. No. 6,958,315 and are generally supplied as clear solutions, 30-50% solids, that are used as pigment or mineral dispersants.

An embodiment of the present invention is also crosspolymers of alkylpolyglucosides and sorbitan esters as sugar-based nonionic dispersants, represented as compounds of the following formulae, and positional isomers thereof, as a mixture:

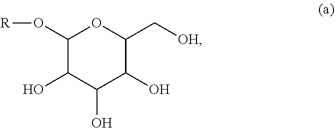

wherein;
R is alkyl having 8 to 22 carbon atoms; and

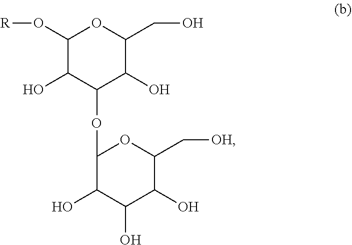

wherein:
R is alkyl having 8 to 22 carbon atoms; and
(c) a sorbitan ester of the following structure:

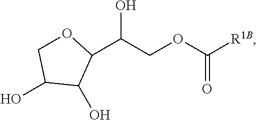

wherein:
R^{1B} is alkyl having 7 to 21 carbons;
a crosslinking agent in water; and optionally a functionalizing agent selected from the group of:

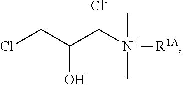

Cl—CH₂—CH(OH)—SO₃M, Cl—CH₂—CH(OH)—SO₄M, Cl—CH₂—CH(OH)CH₂—OP(O)—(OM)₂, and mixtures thereof;
wherein R^{1A} is CH₃—(CH₂)ₙ—,
n is an integer from 0 to 36;
M is a charge balancing group selected from H, Na, K, or NH₄;
and positional isomers thereof.

Crosslinking agents include those described above herein, and are not limited to, 1,3-dichloro-2-propanol and epichlorohydrin.

The crosspolymers of alkylpolyglucosides and sorbitan esters that are the sugar-based nonionic dispersants of the current invention are manufactured by Colonial Chemical, Inc., South Pittsburg, TN 37380, two examples of which are Poly Suga®Mulse D6 and Poly Suga®Mulse D9, both described as sorbitan oleate decylglucoside crosspolymer.

These dispersants are synthesized by the methods outlined in U.S. Pat. No. 8,268,766. The ratio of alkyl polyglucoside monomer to sorbitan ester monomer in the cross polymers can vary from 20:1 to 1:1, respectively, and any range in-between.

An embodiment of the present invention is also sulfonate-modified, phosphate-modified and cationically modified poly-sugar alkyl polyglucoside dispersants, represented by compounds of the following formulae, as a mixture:

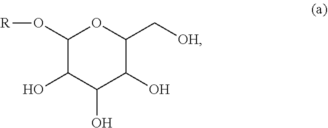

wherein;
R is alkyl having 8 to 22 carbon atoms; and

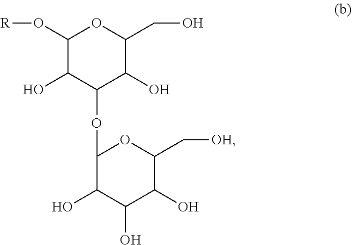

wherein:
R is alkyl having 8 to 22 carbon atoms;
a crosslinking agent; and
a functionalizing agent selected from:

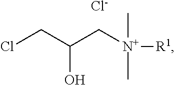

Cl—CH₂—CH(OH)—SO₃M,
Cl—CH₂—CH(OH)—SO₄M,
Cl—CH₂—CH(OH)CH₂—OP(O)—(OM)₂,
and mixtures thereof;
wherein R¹ is CH₃—(CH₂)ₙ—;
n is an integer from 0 to 36;
M is a charge balancing group selected from H, Na, K, or NH₄;
and positional isomers thereof.

Crosslinking agents include those described above herein, and are not limited to, 1,3-dichloro-2-propanol and epichlorohydrin.

These alkyl polyglucosides of the current invention are manufactured by Colonial Chemical, Inc., South Pittsburg, TN 37380, as shown in U.S. Pat. No. 7,507,399. Examples of these alkyl polyglucosides are: sodium hydroxypropyl phosphate decylglucoside crosspolymer (Poly Suga®Phos 1000P), sodium hydroxypropyl phosphate laurylglucoside crosspolymer (PolySuga®Phos 1200P), Sodium hydroxypropyl phosphate cocoglucoside crosspolymer (PolySuga®Phos 8600P), Sodium hydroxypropyl sulfonate butylglucoside crosspolymer (PolySuga®Nate 40P), Sodium hydroxypropyl sulfonate decylglucoside crosspolymer (PolySuga®Nate 100P), Sodium hydroxypropyl sulfonate laurylglucoside crosspolymer (PolySuga®Nate 160P NC), Polyquaternium-78 (Poly Suga®Quat L-1010P), Polyquaternium-80 (Poly Suga®Quat L-1210P) and Polyquaternium-81 (Poly Suga®Quat S-1201P).

Another description of this embodiment is described in U.S. Pat. No. 8,329,633. Thus, poly quaternary functionalized alkyl polyglucosides of the present invention have the following formula:

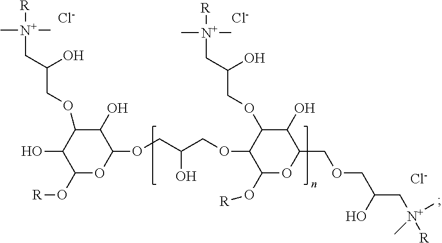

wherein R is an alkyl group having from about 8 to about 22 carbon atoms and n is an integer ranging from 4 to 6; and positional isomers thereof

Another description of this embodiment is described in U.S. Pat. No. 8,262,805. Thus, poly sulfonate functionalized alkyl polyglucosides of the present invention have the following formula:

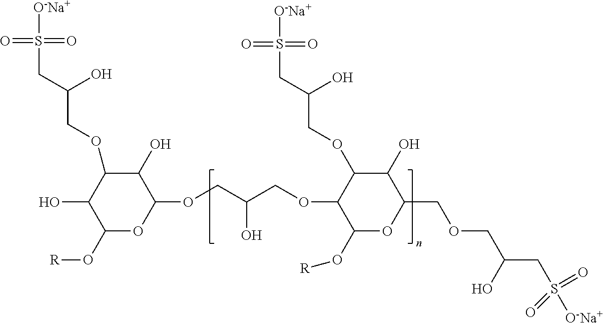

wherein R is an alkyl group having from about 8 to about 22 carbon atoms and n is an integer ranging from 4 to 6; and positional isomers thereof

Another example of this embodiment is described in U.S. Pat. No. 8,287,659. That is, polyphosphate functionalized alkyl polyglucosides of the following formula:

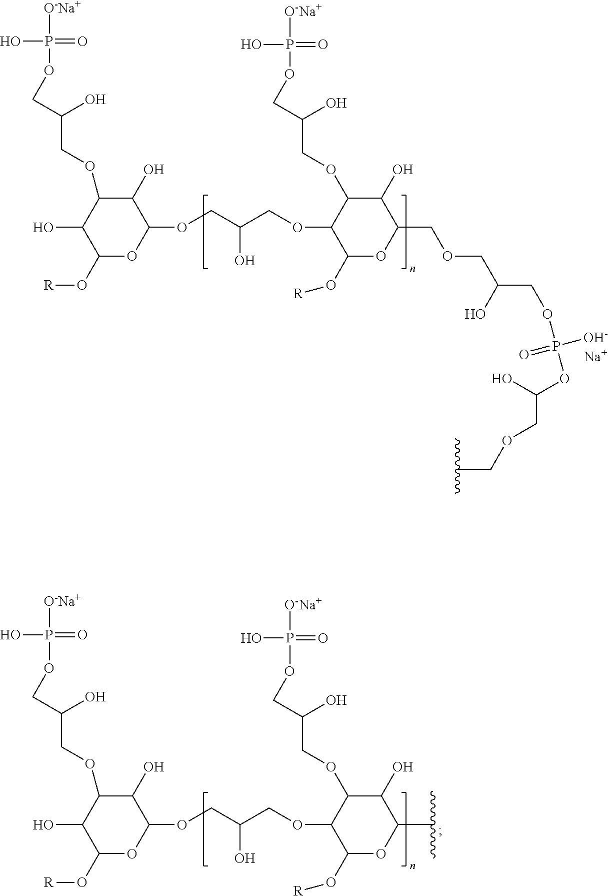

wherein R is an alkyl group having from about 8 to about 22 carbon atoms; and positional isomers thereof.

Another dispersant of the present invention is also described in U.S. Pat. Nos. 8,557,760 and 8,389,457. Quaternary functionalized alkyl polyglucosides of the present invention may have the following representative formula:

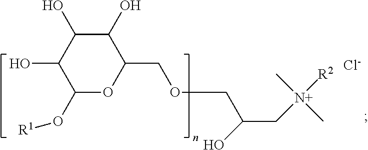

wherein R¹ is an alkyl group having from about 8 to about 22 carbon atoms, and R² is CH₃(CH₂)ₙ, and n is independently an integer from 0-21; and positional isomers thereof. Examples of suitable quaternary functionalized alkyl polyglucosides dispersants include those in the R¹ alkyl moiety contains primarily about 12 carbons, the R² group is CH₃.

Embodiments of the present invention are also sulfosuccinate-modified, alkylpolyglucoside dispersants, represented by compounds of the following formulae, as a mixture:

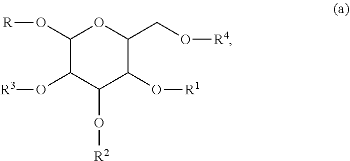

wherein
R is alkyl having 8 to 22 carbon atoms;
R¹, R², R³, and R⁴ are independently selected from —CH₂—CH(OH)—CH₂—R¹², and H,
with the proviso that R¹, R², R³, and R⁴ are not all H;
R¹² is —O—C(O)—CH₂—CH(SO₃M⁺)—C(O)—O⁻M⁺
M is a charge balancing group selected from H, Na, K, or NH₄; and

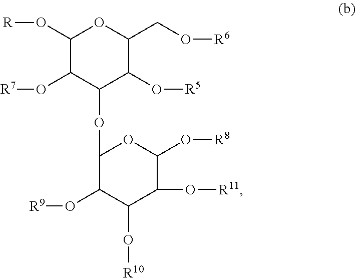

wherein
R is alkyl having 8 to 22 carbon atoms;
R⁵, R⁶, R⁷, R⁸, R⁹, R¹⁰, and R¹¹ are independently selected from —CH₂—CH(OH)—CH₂—R¹², and H,
with the proviso that R⁵, R⁶, R⁷, R⁸, R⁹, R¹⁰, and R¹¹ are not all H;
R¹² is —O—C(O)—CH₂—CH(SO₃M⁺)—C(O)—O⁻M⁺
M is a charge balancing group selected from H, Na, K, or NH₄;
and positional isomers thereof.

The sulfosuccinate-modified PolySuga®alkylpolyglucosides of the current invention are manufactured by Colonial Chemical, Inc., South Pittsburg, TN 37380. These dispersants are synthesized by the methods outlined in U.S. Pat. No. 7,087,571 and are generally supplied as clear solutions, 30-50% solids, that are used as pigment or mineral dispersants.

Another embodiment of the present invention is carboxymethyl-modified, Poly Suga®-alkylpolyglucoside dispersants, represented by the following components, as a mixture:

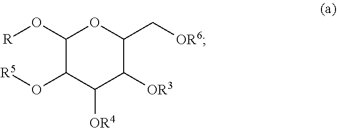

wherein one of R³, R⁴, R⁵, and R⁶ is —CH₂—C(O)—O⁻M⁺ or —C(O)—CH₂—C(O)—O⁻M⁺, with the remaining R groups being H; R is alkyl having 6 to 30 carbon atoms; M is H, Na, or K; and
(b) a 1,3 dicloloro-2-propanol crosslinker;
and positional isomers thereof.

The carboxymethyl-modified Poly Suga®alkyl polyglucosides of the current invention are manufactured by Colonial Chemical, Inc., South Pittsburg, TN 37380, examples of which are Sodium Maleate Decylglucoside Crosspolymer (Poly Suga®Carb DM), Sodium Maleate Laurylglucoside Crosspolymer (Poly Suga®Carb LM) and Sodium Succinate Laurylglucoside Crosspolymer (Poly Suga®Carb LS).

These dispersants are synthesized by the methods outlined in U.S. Pat. No. 7,335,627 and are generally supplied as clear solutions, 40-60% solids, that are used as pigment dispersants.

Another embodiment of the present invention is a citrate-functionalized polymeric alkylglucoside dispersant, represented by the following components, as a mixture:

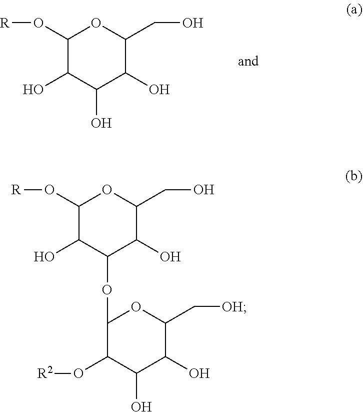

wherein R is an alkyl having 8 to 22 carbons, and R² is:

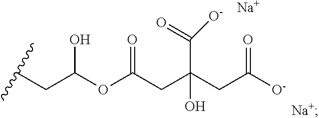

and positional isomers thereof. The citrate-modified alkyl polyglucosides of the current invention are manufactured by Colonial Chemical, Inc., South Pittsburg, TN 37380, an example of which is Disodium Laurylglucosides Hydroxypropyl Citrate (Suga®Citrate L1C)

The functionalized alkylpolyglucosides of the present invention have found wide application mostly in the personal care market in various cleansing products such as shampoos, body washes and facial washes. Additionally, their use in household, industrial and institutional cleaning has been the subject of a number of patents. The Suga®Quats and Poly Suga®Quats dispersants mentioned above have been patented for use in various hard-surface cleaning applications (outlined in U.S. Pat. Nos. 8,557,760; 8,389,457; 8,329,633; 8,877,703; 10,035,975; and 9,474,703 issued to Ecolab USA Inc.). The Suga®Nate and Poly Suga®Nate dispersants mentioned above have also been patented for use in various hard-surface cleaning applications (U.S. Pat. Nos. 8,071,520; 8,216,988; and 8,262,805 issued to Ecolab USA Inc.). The Poly Suga®Glycinates mentioned above have also been patented for use in various hard-surface cleaning applications (U.S. Pat. No. 8,299,099 issued to Ecolab USA Inc.). The Suga®Fax and Poly Suga®Fax dispersants mentioned above have also been patented for use in various hard-surface cleaning applications (U.S. Pat. Nos. 8,216,994; 8,287,659; and 8,969,285 issued to Ecolab USA Inc.). The Poly Suga®Mates dispersants mentioned above have been patented for use in various hard-surface cleaning applications of (U.S. Pat. No. 8,658,584 issued to Ecolab USA Inc.).

As stated above, the present inventors have found the dispersants of the present invention, and combinations thereof, to be useful and beneficial in a pigment or mineral dispersant process. In this embodiment, the dispersant is used to facilitate the wetting, dispersion, and stabilization of a pigment or mineral in a liquid carrier. This generally results in a concentrated pigment slurry or paste.

The pigments suitable for this process are understood to one of ordinary skill in the art. As stated above, examples include of pigments or minerals include, but are not limited to, phthalocyanine pigments, calcium carbonate, titanium dioxide, inorganic clays, iron oxides, ultramarine pigments, han purple, cobalt violet, manganese violet, Persian blue, cobalt blue, cerulean blue, Egyptian blue, manganese blue, azurite, copper carbonate, chrome green, terra verte, primrose yellow, crocoite, aurelian, yellow ochre, zinc chromate, raw umber, raw sienna, carbon black, zinc oxide, alizarin, arylide yellow, azo pigments, diarylide yellow, naphthol red, Indian yellow, indigo dye, orange 15, violet 23, yellow 10, yellow 12, yellow 13, yellow 16, yellow 81, yellow 83, yellow 139, yellow 185, quinacridone, tyrian purple, paris green, or other organic or inorganic pigments.

Another embodiment of the present invention is a method of pigmenting latex or solution-based paints, comprising the dispersants of the present invention. The paints presented in this embodiment may use water, or any other organic liquid as a carrier. In this embodiment, a paint formulation comprises, in part, the dispersants of the present invention to disperse the pigments and/or filler minerals within the paint composition. In this embodiment the dispersant can be added neat to the paint formulation or may be added to the formulation as part of a pigment/mineral slurry.

Another embodiment of the present invention is a method of pigmenting latex or solution-based adhesives, comprising the dispersants of the present invention. The adhesives presented in this embodiment may use water, or any other organic liquid as a carrier. In this embodiment, an adhesive formulation comprises, in part, the dispersants of the present invention to disperse the pigments and/or filler minerals within the paint composition. In this embodiment the dispersant can be added neat to the adhesive formulation or may be added to the formulation as part of a pigment/mineral slurry.

Another embodiment of the present invention is a method of pigmenting latex or solution-based primers, comprising the dispersants of the present invention. The primers presented in this embodiment may use water, or any other organic liquid as a carrier. In this embodiment, a primer formulation comprises the dispersants of the present invention to disperse the pigments and/or filler minerals within the primer composition. In this embodiment the dispersant can be added neat to the primer formulation or may be added to the formulation as part of a pigment/mineral slurry.

Another embodiment of the present invention is a method of pigmenting latex or solution-based textile compounds, comprising the dispersants of the present invention. The textile compounds presented in this embodiment may use water, or any other organic liquid as a carrier. In this embodiment, a textile compound formulation comprises the dispersants of the present invention to disperse the pigments and/or filler minerals within the textile compound composition. In this embodiment the dispersant can be added neat to the textile compound formulation or may be added to the formulation as part of a pigment/mineral slurry.

Another embodiment of the present invention is a method of pigmenting latex or solution-based clear coat compositions, comprising the dispersants of the present invention. The clear coat compositions presented in this embodiment may use water, or any other organic liquid as a carrier. In this embodiment, a clear coat formulation comprises the dispersants of the present invention to disperse the pigments and/or filler minerals within the clear coat composition. In this embodiment the dispersant can be added neat to the clear coat formulation or may be added to the formulation as part of a pigment/mineral slurry.

Another embodiment of the present invention is a method of pigmenting latex or solution-based water-resistant sealers, comprising the dispersants of the present invention. The sealers presented in this embodiment may use water, or any other organic liquid as a carrier. In this embodiment, a sealer formulation comprises the dispersants of the present invention to disperse the pigments and/or filler minerals within the sealer composition. In this embodiment the dispersant can be added neat to the sealer formulation or may be added to the formulation as part of a pigment/mineral slurry.

Another embodiment of the present invention is a method of pigmenting cementitious products, comprising the dispersants of the present invention. These products may include, but are not limited to, concrete, grouts, or mortars. The cementitious products presented in this embodiment may use water, or any other liquid as a carrier, such as a latex. In this embodiment, a cement formulation comprises the dispersants of the present invention to disperse the pigments and/or filler minerals within the cement composition. In this embodiment the dispersant can be added neat to the cement formulation or may be added to the formulation as part of a pigment/mineral slurry.

Another embodiment of the present invention is a method of pigmenting paper products, comprising the dispersants of the present invention. The paper products presented in this embodiment may use water, or any other organic liquid during the process before curing or drying into finished products. In this embodiment, a paper formulation comprises the dispersants of the present invention to disperse the pigments and/or filler minerals within the paper composition. In this embodiment the dispersant can be added neat to the paper formulation or may be added to the formulation as part of a pigment/mineral slurry.

Another embodiment of the present invention is a method of pigmenting or adding filler minerals to solid polymer products, comprising the dispersants of the present invention. The products presented in this embodiment may use water, any other organic liquid as a carrier, or no carrier before curing, drying, or molding to a final product. In this embodiment, a polymer formulation comprises the dispersants of the present invention to disperse the pigments and/or filler minerals within the polymer composition. In this embodiment the dispersant can be added neat to the polymer formulation, added to the formulation as part of a pigment/mineral slurry, or added using any other method known to those skilled in the arts.

Another embodiment of the present invention is a method of pigmenting or adding filler minerals, or functional minerals to personal care products, comprising the dispersants of the present invention. The personal care formulations presented in this embodiment may use water, or any other organic liquid as a carrier. In this embodiment, a personal care formulation comprises the dispersants of the present invention to disperse the pigments and/or filler minerals and/or functional minerals within the cosmetic composition. In this embodiment the dispersant can be added neat to the personal care formulation or may be added to the formulation as part of a pigment/mineral slurry.

Another embodiment of the present invention is a coating composition that comprises (i) at least one derivatized alkyl polyglucoside dispersant and/or alkyl polyglucoside sorbitan ester crosspolymer; (ii) water and/or an organic solvent; a polymer and/or an alkyd; a filler and/or a functional mineral; and at least one colorant or pigment.

In the embodiment, the pigment, filler mineral or functional mineral may be chosen from the following, including, but not limited to, phthalocyanine pigments, calcium carbonate, titanium dioxide, barium sulfate, nepheline syenite, inorganic clays, iron oxides, ultramarine pigments, han purple, cobalt violet, manganese violet, Persian blue, cobalt blue, cerulean blue, Egyptian blue, manganese blue, azurite, copper carbonate, chrome green, terra verte, primrose yellow, crocoite, aurelian, yellow ochre, zinc chromate, raw umber, raw sienna, carbon black, zinc oxide, alizarin, arylide yellow, azo pigments, diarylide yellow, naphthol red, Indian yellow, indigo dye, orange 15, violet 23, yellow 10, yellow 12, yellow 13, yellow 16, yellow 81, yellow 83, yellow 139, yellow 185, quinacridone, tyrian purple, paris green, or other organic or inorganic pigments/filler minerals.

The polymer may be an acrylic polymer, styrene acrylic polymer, vinyl acetate/acrylic polymer, styrene-butadiene polymer, polyisoprene polymer, neoprene polymer, natural rubber polymer, ethylene-vinyl acetate polymer, polyolefin where the individual monomers consist of alpha olefins with carbon chain lengths between 2 and 22, maleic acid-acrylic polymers, maleic acid-styrene polymers, epoxy polymers, polyamides, polyurethanes, polyesters, polyethers, or other polymers suitable for a coating composition.

In other aspects, the polymer is 100% actives. Also, the polymer may be hydrophobic and emulsified in water. In other aspects, the polymer is water soluble and is in an aqueous solution. In other aspects, the polymer is soluble in an organic solvent and is in a solution of said solvent.

The organic solvent may include, but is not limited to, an alcohol, a ketone, an aldehyde, a hydrocarbon, an aromatic hydrocarbon, a halogenated hydrocarbon, a halogenated aromatic hydrocarbon, an acetate, an organic carbonate, dimethyl sulfoxide, an ether, a carboxylic acid, an ester, an amine, or other organic solvent.

The coating may be a multi part system containing monomers that react together once combined, usually, during application.

In embodiments, the monomers may be chosen from glycidyl ethers and amines that react to form polyamines. Also, the monomers may be chosen from glycidyl ethers and alcohols that react to form polyethers. Also, the monomers may be chosen from an amine and an isocyanate and a polyol that react to form polyamides.

The alkyd is the reaction product of a polyol with any or all of the following: (i) one or more organic molecules containing multiple carboxylic acid groups; (ii) one or more anhydride; and/or (iii) one or more fatty acid. Also, the alkyd may be dispersed or emulsified in water. Also, the alkyd may be dissolved in an organic solvent.

### EXAMPLES

The following examples are put forth to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for.

### Example 1: Phthalo Blue

Aqueous pigment dispersions were prepared using phthalo blue as pigment. Several functionalized alkyl polyglucosides or alkyl polyglucoside sorbitan ester crosspolymers were tested in their efficacy as pigment dispersants using the following formula.

| RAW MATERIAL | MASS | PERCENT |
|---|---|---|
| Water | 89.4 | 41.87 |
| Phthalo Blue | 91 | 42.62 |
| Dispersant | 29.85 | 13.98 |
| Bentonite | 1.25 | 0.59 |
| Defoamer | 2 | 0.94 |

In this preparation water, dispersant, and defoamer were first added to a 100 mL paint can and stirred under light agitation until uniform. Once uniform, Phthalo Blue was added slowly under high agitation. Once pigment was all added, the mixture was kept under high agitation to reduce particle size. It is to be noted that deviations from this formula in respect to actual amount of dispersant were made to create a flowable product. Actual percentages and physical property data are detailed in the following table.

| | | % | | HEGMAN | |
|---|---|---|---|---|---|
| DISPERSANT | TSC | DISPERSANT | VISCOSITY | (μm) | STATUS |
| Poly Suga ®Mulse D9 | 52.15 | 9.78 | 165 | 0 | PASS* |
| Suga ®Fax D10 | 48.56 | 17.36 | 165 | <5 | PASS |
| Suga ® Nate 160 NC | 48.73 | 15.03 | 185 | <3 | PASS* |
| Poly Suga ® Nate | 48.82 | 13.98 | 200 | <8 | PASS* |
| 100P | | | | | |

*In all cases the phthalo blue pigment was successfully dispersed. In the experiments marked with (*) the dispersions did settle within a 14-day window. Those skilled in the art realize that adjusting other formulation parameters can result in stable dispersions. All other dispersions were stable without further adjustment.
[Poly Suga ®Mulse D9 (Sorbitan Oleate Decylglucoside Crosspolymer), Suga ®Fax D10NC (Sodium Decylglucosides Hydroxypropyl Phosphate), Suga ®Nate 160NC (Sodium Laurylglucosides Hydroxypropylsulfonate, and PolySuga ®Nate 100P (Sodium Hydroxypropyl Sulfonate Decylglucoside Crosspolymer) are manufactured and sold by Colonial Chemical, Inc. located in South Pittsburg, TN].

### Example 2: Toluidine Red

Aqueous pigment dispersions were prepared using toluidine red as pigment. Several functionalized alkyl polyglucosides or alkyl polyglucoside sorbitan ester crosspolymers were tested in their efficacy as pigment dispersants using the following formula.

| RAW MATERIAL | MASS | PERCENT |
|---|---|---|
| Water | 89.4 | 41.87 |
| Toluidine Red | 91 | 42.62 |
| Dispersant | 29.85 | 13.98 |
| Bentonite | 1.25 | 0.59 |
| Defoamer | 2 | 0.94 |

In this preparation water, dispersant, and defoamer were first added to a 100 mL paint can and stirred under light agitation until uniform. Once uniform, Toluidine Red was added slowly under high agitation. Once pigment was all added, the mixture was kept at under high agitation until particle size was reduced to less than 10 micrometers. It is to be noted that deviations from this formula in respect to actual amount of dispersant were made to create a flowable product. Actual percentages and physical property data are detailed in the following table.

| | | % | | HEGMAN | |
|---|---|---|---|---|---|
| DISPERSANT | TSC | DISPERSANT | VISCOSITY | (μm) | STATUS |
| Poly Suga ®Mulse D9 | 49.72 | 19.45 | 270 | 0 | PASS |
| Suga ®Fax D10 | 45.69 | 22.7 | 245 | 0 | PASS |
| Suga ®Nate 160 NC | 45.46 | 22.63 | 310 | 0 | PASS |
| Poly Suga ®Nate | 45.72 | 18.92 | 255 | 0 | PASS* |
| 100P | | | | | |

*In all cases the toluidine red pigment was successfully dispersed. In the experiment marked with (*) the dispersion did settle within a 14-day window. Those skilled in the art realize that adjusting other formulation parameters can result in stable dispersions. All other dispersions were stable without further adjustment.

### Example 3: Yellow 170

Aqueous pigment dispersions were prepared using Yellow 170 as pigment. several functionalized alkyl polyglucosides or alkyl polyglucoside sorbitan ester crosspolymers were tested in their efficacy as pigment dispersants using the following formula.

| RAW MATERIAL | MASS | PERCENT |
|---|---|---|
| Water | 89.4 | 41.87 |
| Yellow 170 | 91 | 42.62 |
| Dispersant | 29.85 | 13.98 |
| Bentonite | 1.25 | 0.59 |
| Defoamer | 2 | 0.94 |

In this preparation water, dispersant, and defoamer were first added to a 100 mL paint can and stirred under light agitation until uniform. Once uniform, Yellow 170 was added slowly under high agitation. Once pigment was all added, the mixture was kept at under high agitation until particle size was reduced to less than 10 micrometers. It is to be noted that deviations from this formula in respect to actual amount of dispersant were made to create a flowable product. Actual percentages and physical property data are detailed in the following table.

| | | % | | HEGMAN | |
|---|---|---|---|---|---|
| DISPERSANT | TSC | DISPERSANT | VISCOSITY | (μm) | STATUS |
| Poly Suga ®Mulse D9 | 50.91 | 18.98 | 165 | 0 | PASS |
| Suga ®Fax D10 | 44.36 | 15.25 | 165 | 0 | PASS* |
| Suga ®Nate 160 NC | 46.54 | 14.65 | 185 | 0 | PASS |
| Poly Suga ®Nate | 46.41 | 14.65 | 200 | 0 | PASS |
| 100P | | | | | |

*In all cases the Yellow 170 pigment was successfully dispersed. In the experiment marked with (*) the dispersions did settle within a 14-day window. Those skilled in the art realize that adjusting other formulation parameters can result in stable dispersions. All other dispersions were stable without further adjustment.

### Example 4: Titanium Dioxide

Aqueous pigment dispersions were prepared using titanium dioxide as pigment. several functionalized alkyl polyglucosides or alkyl polyglucoside sorbitan ester crosspolymers were tested in their efficacy as pigment dispersants using the following formula.

| RAW MATERIAL | MASS | PERCENT |
|---|---|---|
| Water | 89.1 | 35.31 |
| Titanium Dioxide | 150 | 59.44 |
| Dispersant | 10 | 3.96 |
| Bentonite | 1.25 | 0.50 |
| Defoamer | 2 | 0.79 |

In this preparation water, dispersant, and defoamer were first added to a 100 mL paint can and stirred under light agitation until uniform. Once uniform, Titanium Dioxide was added slowly under high agitation. Once pigment was all added, the mixture was kept at under high agitation until particle size was reduced to less than 10 micrometers. It is to be noted that deviations from this formula in respect to actual amount of dispersant were made to create a flowable product. Actual percentages and physical property data are detailed in the following table.

| | | % | | HEGMAN | |
|---|---|---|---|---|---|
| DISPERSANT | TSC | DISPERSANT | VISCOSITY | (μm) | STATUS |
| Poly Suga ®Phos 1000P | 59.37 | 2.05 | 495 | <5 | PASS |
| Suga ®Nate 160 NC | 61.82 | 1.92 | 195 | <5 | PASS |
| Poly Suga ®Nate 160P | 65.81 | 1.93 | 575 | <5 | PASS |
| Polyacrylic Acid Control | 62.07 | 7.71 | 265 | <5 | FAIL* |

*In all cases the Titanium Dioxide was successfully dispersed. In the control experiment marked with (*) the dispersion did settle within a 14-day window. All other dispersions were stable without further adjustment.
Poly Suga ®Phos 1000P (Sodium Hydroxypropylphosphate Decylglucoside Crosspolymer) and PolySuga ®Nate 160P (Sodium Hydroxypropyl Sulfonate Laurylglucoside Crosspolymer) are manufactured and sold by Colonial Chemical, Inc., South Pittsburg, TN.

### Example 5: Yellow Iron Oxide

Aqueous pigment dispersions were prepared using Yellow Iron Oxide as pigment. several functionalized alkyl polyglucosides or alkyl polyglucoside sorbitan ester crosspolymers were tested in their efficacy as pigment dispersants using the following formula.

| RAW MATERIAL | MASS | PERCENT |
|---|---|---|
| Water | 89.1 | 35.31 |
| Yellow Iron Oxide | 150 | 59.44 |
| Dispersant | 10 | 3.96 |
| Bentonite | 1.25 | 0.50 |
| Defoamer | 2 | 0.79 |

In this preparation water, dispersant, and defoamer were first added to a 100 mL paint can and stirred under light agitation until uniform. Once uniform, Yellow Iron Oxide was added slowly under high agitation. Once pigment was all added, the mixture was kept at under high agitation until particle size was reduced to less than 10 micrometers. It is to be noted that deviations from this formula in respect to actual amount of dispersant were made to create a flowable product. Actual percentages and physical property data are detailed in the following table.

| | | % | | HEGMAN | |
|---|---|---|---|---|---|
| DISPERSANT | TSC | DISPERSANT | VISCOSITY | (μm) | STATUS |
| Poly Suga ®Phos 1000P | 54.05 | 4.3 | 150 | <5 | PASS |
| Suga ®Nate 160 NC | 53.89 | 4.3 | 135 | <5 | PASS |
| Poly Suga ®Nate 160P | 54.12 | 4.3 | 142 | <5 | PASS |
| Polyacrylic Acid Control | 54.58 | 4.3 | 175 | <5 | FAIL* |

*In all cases the Yellow Iron Oxide was successfully dispersed. In the control experiment marked with (*) the dispersion did settle within a 14-day window. All other dispersions were stable without further adjustment.

### Example 6: Dispersant Demand Viscosity Curves

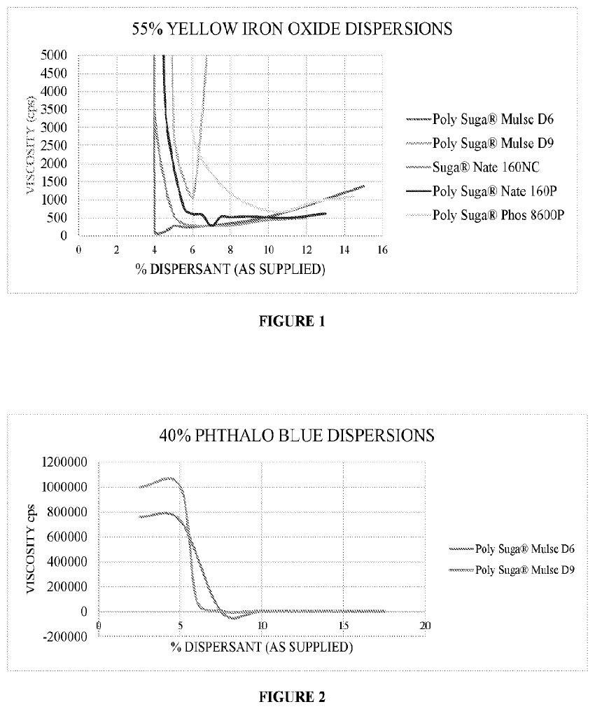

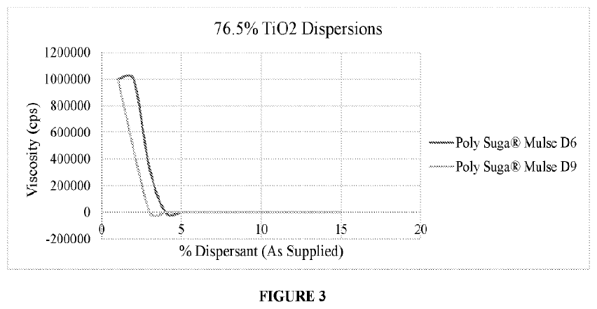

Dispersant demand viscosity curves were created to find the optimum usage levels of several dispersants outlined within this patent. In this preparation water, dispersant, and defoamer were first added to a 1000 mL beaker and stirred under light agitation until uniform. Once uniform, pigment was added slowly under high agitation using a rotor stator mixer, Ross Model HSM-100LSI-I Laboratory High Shear Mixer. Once pigment was all added, the mixture was kept at under high agitation until particle size was reduced to less than 10 micrometers. Three different pigments were chosen for this study to evaluate the efficacy of the dispersants over a broad spectrum of pigment chemistries: yellow iron oxide, phthalo blue and titanium dioxide. The viscosity curves for these three pigments in various derivatized alkyl polyglucosides and alkyl polyglucosides (APG) crosspolymers are shown in FIGS. 1 through 3 below. The data for those curves in listed in Tables 1 through 3.

TABLE 1
Dispersant Demand Viscosity Curve data for Yellow Iron Oxide in various derivatized
alkyl polyglucosides and APG crosspolymers

| PERCENT | Poly Suga ® | Poly Suga ® | Suga ® Nate | Poly Suga ® | Poly Suga ® |
|---|---|---|---|---|---|
| DISPERSANT | Mulse D6 | Mulse D9 | 160NC | Nate 160P | Phos 8600P |
| 1 | — | — | 1000000+ | — | — |
| 2 | 1000000+ | 1000000+ | 1000000+ | 1000000+ | 1000000+ |
| 3 | 300000 | 255000 | 1000000+ | — | — |
| 4 | 160 | 40000 | 3750 | 27500 | 1000000+ |
| 4.5 | — | — | 1580 | 4100 | — |
| 5 | 280 | 3000 | 585 | — | 375000 |
| 5.5 | — | — | 340 | 820 | 57000 |
| 6 | 240 | 1050 | 290 | 605 | 2950 |
| 6.5 | — | — | 270 | 580 | — |
| 7 | — | 6600 | 280 | 275 | 1740 |
| 8 | — | — | 290 | 516 | 1000 |
| 10 | 530 | — | 435 | — | 660 |
| 11 | — | — | — | 500 | — |
| 13 | — | — | — | 615 | 950 |
| 15 | 1370 | — | — | — | 1100 |

TABLE 2
Dispersant Demand Viscosity Curve data for Phthalo
Blue in Alkyl Poly Glucoside crosspolymers

| PERCENT DISPERSANT | Poly Suga ® Mulse D6 | Poly Suga ® Mulse D9 |
|---|---|---|
| 2.5 | 762000 | 1000000+ |
| 5 | 729000 | 1000000+ |
| 6 | 330000 | 89000 |
| 7.5 | 325 | 1000 |
| 10 | 45 | 1035 |
| 15 | 700 | 1700 |
| 17.5 | 2200 | — |

TABLE 3
Dispersant Demand Viscosity Curve data for Titanium Dioxide
in Alkyl Poly Glucoside crosspolymers

| PERCENT DISPERSANT | Poly Suga ® Mulse D6 | Poly Suga ® Mulse D9 |
|---|---|---|
| 1 | 1000000+ | 1000000+ |
| 2 | 1000000+ | — |
| 3 | 330000 | 600 |
| 4 | 250 | 375 |
| 5 | 350 | 520 |
| 6 | 810 | 535 |
| 7 | 1370 | — |
| 10 | 2700 | 560 |
| 15 | | 1100 |

### Example 7: Stability Studies

The following is exemplary of how stability studies were conducted on the pigment/mineral dispersions in the various surfactants of this invention. In general, it was found the derivatized alkylpolyglucoside improved stability against settling and sedimentation of pigment dispersion.

### Example 8: Stability of Napthol Red AS dispersions

Several dispersions of Napthol Red AS were prepared using the percentages in Table 4 in order to test their long-term stability. In these preparation water, dispersant, and defoamer were first added to a 1000 mL beaker and stirred under light agitation until uniform. Once uniform, pigment was added slowly under high agitation. Once pigment was all added, the mixture was kept at under high agitation using a rotor stator mixer until particle size was reduced to less than 10 micrometers. Each sample was tested for settling after 30 days of storage at room temp and at 50° C.

TABLE 4
Stability of Napthol Red AS

| | | | Poly | Poly | | | 30 | 30 Day |
|---|---|---|---|---|---|---|---|---|
| Sample | | | Suga ®Mulse | Suga ®Mulse | Napthol | | Stability | Stability |
| ID | Water | Defoamer | D6 | D9 | Red AS | Viscosity | RT | 50° C. |
| Sample | 52.7 | 1.4 | 8 | 0 | 37.9 | 185 | PASS | PASS |
| 1 | | | | | | | | |
| Sample | 52.7 | 1.4 | 0 | 8 | 37.9 | 210 | PASS | PASS |
| 2 | | | | | | | | |

The examples above are exemplary and clearly illustrate the dispersing properties of derivatized alkyl poly glucosides and crosspolymers of alkyl polyglucosides and sorbitan esters for pigments, filler minerals, or functional minerals. It is herein proposed that the class of natural surfactants derived from alkylpoly glucosides can be used for the dispersion of pigments within a liquid medium to form dispersions of low viscosity and high concentrations.

As used herein, the singular forms “a,” “an” and “the” include plural referents unless the context clearly dictates otherwise. Thus, for example, reference to “a surfactant” includes mixtures of two or more such dispersants.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present invention includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers. Additionally, unless expressly described as “unsubstituted”, all substituents can be substituted or unsubstituted.

The invention thus being described, it will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only.

1. A composition, comprising:
a. at least one derivatized alkyl polyglucoside, and/or an alkyl polyglucoside sorbitan ester crosspolymer dispersant in a range of 0.01-50%;
b. an organic solvent or water, in a range from 20-80% by weight;
c. at least one pigment, filler mineral, or functional mineral making up 20-80% of the dispersion.

2. A composition, comprising:
a. at least one derivatized alkyl polyglucoside, and/or alkyl polyglucoside sorbitan ester crosspolymer dispersant in a range of 0.01-50%;
b. an organic solvent, in a range from 20-80% by weight;
c. at least one pigment, filler mineral, or functional mineral making up 20-80% of the dispersion

3. A composition of claim 1, wherein the pigment, filler mineral or functional mineral is chosen from the following, including, but not limited to, phthalocyanine pigments, calcium carbonate, titanium dioxide, barium sulfate, nepheline syenite, inorganic clays, iron oxides, ultramarine pigments, han purple, cobalt violet, manganese violet, Persian blue, cobalt blue, cerulean blue, Egyptian blue, manganese blue, azurite, copper carbonate, chrome green, terra verte, primrose yellow, crocoite, aurelian, yellow ochre, zinc chromate, raw umber, raw sienna, carbon black, zinc oxide, alizarin, arylide yellow, azo pigments, diarylide yellow, naphthol red, Indian yellow, indigo dye, orange 15, violet 23, yellow 10, yellow 12, yellow 13, yellow 16, yellow 81, yellow 83, yellow 139, yellow 185, quinacridone, tyrian purple, paris green, or other organic or inorganic pigments/filler minerals.

4. The composition of claim 1, wherein the derivatized alkyl polyglucoside is chosen from a carboxymethyl derivatized alkyl polyglucoside, a quaternary derivatized alkyl poylglucoside, a sulfonate derivatized alkyl polyglucoside, a phosphate derivatized alkyl polyglucoside, a sulfosuccinate derivatized alkyl polyglucoside, a glycinate derivatized alkyl polyglucoside, and a citrate derivatized alkyl polyglucoside.

5. The composition of claim 1, wherein the derivatized alkyl polyglucoside is chosen from a polysulfonate derivatized alkyl polyglucoside, polyphosphate derivatized alkyl polyglucoside, polyquaternary derivatized alkyl polyglucoside, polycarboxylated derivatized alkyl polyglucoside, and a polycitrate derivatized alkyl polyglucoside.

6. The composition of claim 1, wherein the alkyl polyglucoside crosspolymer is a sorbitan ester alkylglucoside crosspolymer including sorbitan oleate decylglucoside crosspolymer.

7. The composition of claim 1, wherein the composition is ethylene oxide and 1,4-dioxane free.

8. The composition of claim 1, wherein the derivatized polyglucoside dispersant of the present invention comprises a monosaccharide unit, a disaccharide unit, a linker, and a functionalizing agent.

9. The composition of claim 1, wherein the derivatized polyglucoside dispersant of the present invention comprises a monosaccharide unit, a disaccharide unit, a crosslinking agent, and a functionalizing agent.

10. The composition of claim 1, wherein the derivatized alkyl polyglucoside dispersant is a composition that includes the following, as a mixture: 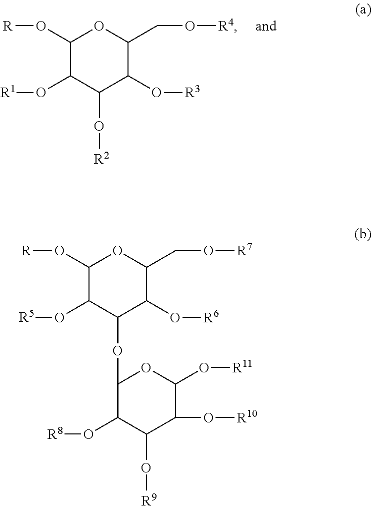
wherein:
R is an alkyl chain having 8 to 22 carbon atoms;
R¹, R², R³, R⁴, R⁵, R⁶, R⁷, R⁸, R⁹, R¹⁰, and R¹¹ are independently selected from the group consisting of: —CH₂—C(O)—O⁻M⁺, or —C(O)CH₂—C(O)—O⁻M⁺ 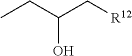
and H, with the proviso that R¹-R¹¹ are not all H;
R¹² is selected from the group consisting of:
—OH, —SO₃⁻M⁺, and —SO₄⁻²M⁺, —O—P(O)—(OM)₂,
—N(CH₃)₂—R^{1A}, —O—C(O)—CH₂—OH(SO₃⁻M⁺)—C(O)—O⁻M⁺, 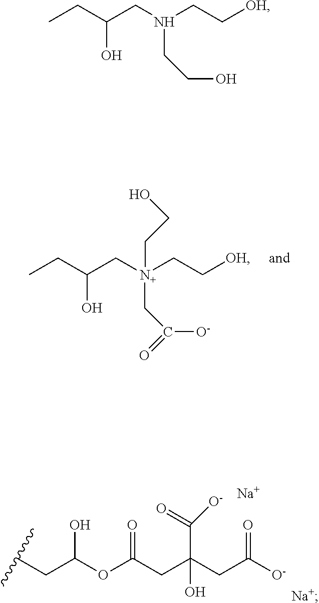
R^{1A} is CH₃—(CH₂)ₙ—;
M is a charge balancing group selected from H, Na, K, or NH₄⁺; and
n is an integer from 0-36;
and positional isomers thereof.

11. The composition of claim 1, wherein the derivatized alkyl polyglucoside dispersant is a composition that includes the following, as a mixture: 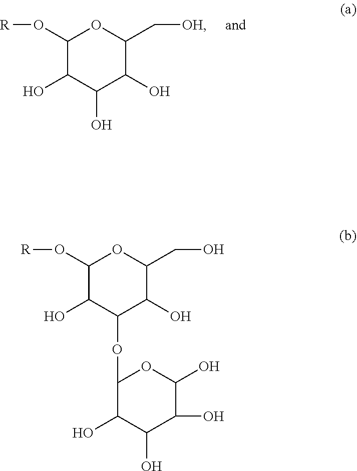
wherein:
R is an alkyl chain having 8 to 22 carbon atoms;
a crosslinking agent; and
a functionalizing agent selected from: 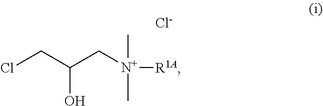
Cl—CH₂—CH(OH)—SO₃M, Cl—CH₂—CH(OH)—SO₄M, Cl—CH₂—CH(OH)—CH₂—OP(O)—(OM)₂, and combinations thereof, wherein
R^{1A} is CH₃(CH₂)ₙ—;
(ii) —Cl—CH₂—C(O)—O⁻²M⁺, 2-halocarboxylic acid, α, β-unsaturated carboxylic acid, cyclic carboxylic acid anhydride, and combinations thereof; 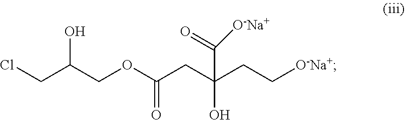
M is a charge balancing group selected from H, Na, K, or NH₄⁺; and
n is an integer from 0-36;
and positional isomers thereof.

12. The composition of claim 11, wherein the crosslinking agent is 1,3-dichloro-2-propanol or epichlorohydrin.

13. The composition of claim 1, wherein the alkyl polyglucoside sorbitan ester crosspolymer dispersant is a composition that includes the following, as a mixture: 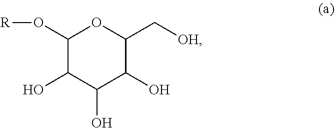
wherein;
R is alkyl having 8 to 22 carbon atoms; and 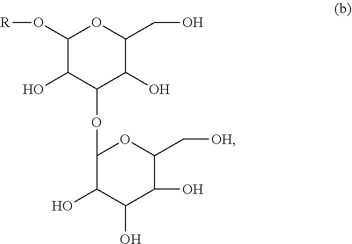
wherein:
R is alkyl having 8 to 22 carbon atoms; and
(c) a sorbitan ester of the following structure: 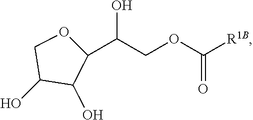
wherein:
R^{1B} is alkyl having 7 to 21 carbons;
a crosslinking agent in water; and optionally a functionalizing agent selected from the group of: 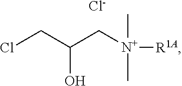
Cl—CH₂—CH(OH)—SO₃M, Cl—CH—2—CH(OH)—SO₄M, Cl—CH₂—CH(OH)CH₂—OP(O)—(OM)₂, and mixtures thereof;
wherein R^{1A} is CH₃—(CH₂)ₙ—,
n is an integer from 0 to 36;
M is a charge balancing group selected from H, Na, K, or NH₄;
and positional isomers thereof.

14. The composition of claim 13, wherein the crosslinking agent is 1,3-dichloro-2-propanol or epichlorohydrin.

15. The composition of claim 1, further comprising a co-dispersant.

16. The composition of claim 15, wherein the co-dispersant is a derivatized alkyl polyglucoside or alkyl polyglucoside sorbitan ester crosspolymer dispersant that is ethylene oxide and 1,4-dioxane free.

17. The composition of claim 14, wherein the co-dispersant is selected from alkyl polyglucosides, alkyl polyglucoside sorbitan ester crosspolymers, fatty alcohol ethoxylates, alkylphenol ethoxylates, phosphate esters of fatty alcohols, phosphate esters of fatty alcohol ethoxylates, phosphate esters of alkylphenol ethoxylates, sulfates of fatty alcohols, sulfates of fatty alcohol ethoxylates, sulfates of alkylphenol ethoxylates, sulfonates of fatty alcohols, sulfonates of fatty alcohol ethoxylates, sulfonates of alkylphenol ethoxylates, ethoxylated terpenes, phosphate esters of ethoxylated terpenes, sulfates of ethoxylated terpenes, sulfonates of ethoxylated terpenes, sulfosuccinates, quaternary amines, betaines, carboxymethylated fatty alcohols, carboxymethylated fatty alcohol ethoxylates, carboxymethylated alkylphenol ethoxylates, polyacrylic acid polymers, polymethacrylic acid polymers, gemini surfactants, or tertiary amine surfactants.

18. The composition of claim 1, wherein the derivatized alkylpolyglucoside or alkyl polyglucoside sorbitan ester crosspolymer improves stability against settling and sedimentation of pigment dispersion”

19. A coating composition, comprising:
(i) at least one derivatized alkyl polyglucoside dispersant and/or alkyl polyglucoside sorbitan ester crosspolymer;
(ii) water, or organic solvent;
(iii) a polymer or alkyd;
(iv) a filler or functional mineral; and
(v) at least one colorant or pigment.

20. The composition of claim 19, wherein the pigment, filler mineral or functional mineral is chosen from the following, including, but not limited to, phthalocyanine pigments, calcium carbonate, titanium dioxide, barium sulfate, nepheline syenite, inorganic clays, iron oxides, ultramarine pigments, han purple, cobalt violet, manganese violet, Persian blue, cobalt blue, cerulean blue, Egyptian blue, manganese blue, azurite, copper carbonate, chrome green, terra verte, primrose yellow, crocoite, aurelian, yellow ochre, zinc chromate, raw umber, raw sienna, carbon black, zinc oxide, alizarin, arylide yellow, azo pigments, diarylide yellow, naphthol red, Indian yellow, indigo dye, orange 15, violet 23, yellow 10, yellow 12, yellow 13, yellow 16, yellow 81, yellow 83, yellow 139, yellow 185, quinacridone, tyrian purple, Paris green, or other organic or inorganic pigments/filler minerals.

21. The composition of claim 19, wherein the polymer is an acrylic polymer, styrene acrylic polymer, vinyl acetate/acrylic polymer, styrene-butadiene polymer, polyisoprene polymer, neoprene polymer, natural rubber polymer, ethylene-vinyl acetate polymer, polyolefin where the individual monomers consist of alpha olefins with carbon chain lengths between 2 and 22, maleic acid-acrylic polymers, maleic acid-styrene polymers, epoxy polymers, polyamides, polyurethanes, polyesters, polyethers, or other polymers suitable for a coating composition.

22. The composition of claim 21, wherein the polymer is 100% actives.

23. A composition of claim 19, wherein the polymer is hydrophobic and is emulsified in water.

24. The composition of claim 19, wherein the polymer is water soluble and is in an aqueous solution.

25. The composition of claim 19, wherein the polymer is soluble in an organic solvent and is in a solution of said solvent.

26. The composition of claim 19 where the organic solvent includes, but is not limited to, an alcohol, a ketone, an aldehyde, a hydrocarbon, an aromatic hydrocarbon, a halogenated hydrocarbon, a halogenated aromatic hydrocarbon, an acetate, an organic carbonate, dimethyl sulfoxide, an ether, a carboxylic acid, an ester, an amine, or other organic solvent.

27. The composition of claim 19, wherein the coating is a multi-part system containing monomers that react together once combined, usually, during application.

28. The composition of claim 27 where the monomers comprise glycidyl ethers and amines that react to form polyamines.

29. The composition of claim 27 where the monomers comprise glycidyl ethers and alcohols that react to form polyethers.

30. The composition of claim 27, where the monomers comprise an amine and an isocyanate and a polyol that react to form polyamides.

31. The composition of claim 19, where the alkyd is the reaction product of a polyol with any or all of the following: one or more organic molecules containing multiple carboxylic acid groups; one or more anhydride; and/or one or more fatty acid.

32. The composition of claim 19, where the alkyd is dispersed or emulsified in water.

33. The composition of claim 19, where the alkyd is dissolved in an organic solvent.

34. The composition of claim 33, where the organic solvent includes, but is not limited to, an alcohol, a ketone, an aldehyde, a hydrocarbon, an aromatic hydrocarbon, a halogenated hydrocarbon, a halogenated aromatic hydrocarbon, an acetate, an organic carbonate, dimethyl sulfoxide, an ether, a carboxylic acid, an ester, an amine, or other organic solvent.
